(19) United States
(12) Patent Application Publication
(10) Pub. No.: US 2006/0141046 A1
(43) Pub. Date: Jun. 29, 2006

(54) Particles for the delivery of active agents

(75) Inventor: Maurizio V. Cattaneo, Quincy, MA (US)

Correspondence Address: FISH & NEAVE IP GROUP;ROPES & GRAY LLP, ONE INTERNATIONAL PLACE, BOSTON, MA, 02110-2624, US

(73) Assignee: IVREA Pharmaceuticals, Inc., Quincy, MA (US)

(21) Appl. No.: 11/283,064

(22) Filed: Nov. 17, 2005

Related U.S. Application Data
(63) Continuation-in-part of application No. 11/123,958, filed on May 6, 2005.
Application No. 11/123,958 is a continuation-in-part of application No. 10/839,907, filed on May 6, 2004.
(60) Provisional application No. 60/634,885, filed on Dec. 9, 2004.

(51) Int. Cl. A61K 9/14

(52) U.S. Cl. 424/489

(57) ABSTRACT

Formulations of active agent particles of less than 100 microns in a droplet of dispersant, which is coated with a matrix of cationic and anionic polymers, are efficient vehicles for delivering active agents to tissues such as skin and mucosal membranes. Such formulations are able to deliver compounds to skin with little associated irritation. Prior art topical formulations typically have the disadvantage of causing significant skin irritation.

### RELATED APPLICATIONS

This application is a continuation-in-part of U.S. application Ser. No. 11/123,958, filed May 6, 2005, which is a continuation-in-part of U.S. application Ser. No. 10/839,907, filed May 6, 2004, and claims the benefit of U.S. Provisional Application No. 60/634,885, filed Dec. 9, 2004. The entire teachings of the above applications are incorporated herein by reference.

### STATEMENT REGARDING FEDERALLY-SPONSORED RESEARCH

This work was sponsored in part by NIH Grant 2R44 CA086653. The Government has certain rights in this invention.

### BACKGROUND OF THE INVENTION

Topical retinoids such as retinoic acid have been used to treat skin conditions such as acne, actinic keratosis, psoriasis, skin cancers and photodamage and chemoprevention of melanoma [Griffiths et al., N Eng J Med 329:530-534 (1993); Halpern et al., In: Advances in the biology and treatment of cutaneous melanoma, Boston, Mass., Nov. 6-7 (1998); Kligman, J Am Acad Dermatol 39:S2-S7 (1998); Stam-Postuma, Melanoma Research 8:539-48 (1998); Varani et al. J Inv Dermatol 114:480-486 (2000)].

One side effect of topical retinoic acid for treating skin ailments is increased irritation. Topical tretinoin (all-trans-retinoic acid, ATRA, retinoic acid) induces irritation in 90% of patients (Gilchrest, J Am Acad Dermatol 36:S27-S36 (1997)], and other side effects include patchy erythema, localized swelling, xerosis, and scaling. Irritation has been attributed, in part, by an overload of the tretinoin dependent pathways with non-physiological amounts of exogenous tretinoin in the skin (Siegenthaler et al., in: Retinoids: From Basic Science To Clinical Applications, M. A. Livrea and G. Vidali (eds), Birkhauser Verlag, Basel, Switzerland, pp. 329-335, (1994)). For example, compared to oral administration, topical delivery of retinoic acid increases the concentration of retinoic acid in the dermal compartment 10- to 100-fold (Lehman et al., J Invest Dermatol 91:56-61 (1988)). This irritation may be the reason for discontinuation of treatment for approximately 50% of patients (Stam-Postuma et al., Melanoma Research 8:539-48 (1998)). This high incidence of irritation, leading to poor compliance, can preclude its use. Any means to reduce irritation is therefore seen as a very desirable attribute of any topical formulation. In the prior art, entrapment of retinoic acid in porous microspheres (Microsponge®) to slow down its release into the skin layers, resulted in a reduction of the level of irritation by controlling the release of the active into the skin (Won et al., U.S. Pat. No. 5,955,109 (1999)). However, formulas containing this delivery system tend to deposit a fine dry residue on the skin surface which may not be cosmetically acceptable.

In addition to being quite irritating, there are problems with the topical administration of retinoids and compounds such as Vitamin D3 due to their insolubility in water and their photolability. The low solubility limits the incorporation of these drugs into acceptable vehicles and their photolability may render topically applied drugs ineffective. The insolubility problems mean that these drugs cannot be administered topically without additives and solubilizing agents, which are generally irritating. When a person applies these drugs topically they have to cross the stratum corneum before they can get to the target tissue, which are the epidermal and dermal layers. Any further penetration of the active into the systemic circulation should be avoided since this triggers the release of certain cytokines such as IL-1α and results in a secondary irritation response.

Because of the irritation caused by these actives, there has been a demand to replace conventional topical formulations (e.g., gels, creams and lotions) for years. The problem has been how to mix an insoluble drug in a carrier solution without using potentially irritating additives and/or solubilizing agents. In addition, there is a problem of how to “mask” the drug in an agent that would stabilize the drug and be more easily tolerated by the patient.

Although chitosan has been contemplated as an ingredient in topical formulations, previous formulations have not remedied all of the problems described above.

In Grandmontagne et al. (U.S. Pat. No. 6,242,099 (2001)), microcapsules made of chitin or a chitin derivative enveloping a hydrophobic substance were made using an anionic surfactant and chitosan. The anionic surfactant plays the dual role of emulsifying a hydrophobic substance as well as precipitating the chitosan polymer. The chitosan was further processed by crosslinking or formed into chitin by acetylation. In this invention the formation of microcapsules requires the use of anionic surfactants which may cause adverse skin reactions such as erythema and edema. The use of surfactants to precipitate chitosan was also disclosed in German patent applications DE 19712978 A1 and DE 19756452 A1, which describe microspheres made by mixing chitosans or chitosan derivatives with oil bodies and precipitating these mixtures into alkaline surfactant solutions.

Garces et al. describe microcapsules of 0.1 mm to 5 mm in diameter (U.S. Publication No. 2003/0064106) made by encapsulating an emulsion of the active ingredient with an anionic polymer followed by chitosan. These microcapsules were obtained by a method that includes emulsifiers to form the initial emulsion and solubilize the active ingredient.

Thus, prior art encapsulation methods relied on surfactants and/or emulsifiers as a critical step in the making of the chitosan-based microparticulates. These surfactants, especially the anionic surfactants can contribute to increased skin irritation and other adverse skin reactions. In addition, some of these encapsulation procedures leave a cosmetically unacceptable residue after topical application. Thus, a formulation that overcomes these problems is needed.

### BRIEF DESCRIPTION OF THE INVENTION

The present invention is based on the discovery that water insoluble active agents can be delivered in the form of microparticles or nanoparticles (generically, particles) that are suitable for administration (e.g., topical, transdermal, transmucosal administration). That is, having improved transport properties to or through skin or mucosal surfaces and/or reduced irritation at the site of administration. The subject compositions obviate the need for administration of insoluble active agents (e.g. retinoic acid) in an emulsion containing solvent or surfactants that cause irritation, as for example, ethanol and polyethoxylated castor oil (see for example Technical Bulletin ME 142e, Tretinoin for the Pharmaceutical Industry, October 1998, BASF Corporation). Relative to the active agent alone or in these other formulations, the present systems reduce or eliminate adverse skin reactions such as erythema and swelling. Accordingly, compositions of the invention can deliver active agents that otherwise cause reactions, such as retinoic acid, retinol and calcipotriene.

In certain embodiments, the invention provides a composition for administration of a water insoluble or slightly water soluble active agent, which includes particles having a mean diameter of 100 microns or less. In certain embodiments, the particles are nanoparticles, having a mean diameter of less than 1 micron, such as from 10 nm to 500 nm or from 20 mn to 300 nm. The particles include an inner core containing the active agent (e.g., as primarily solid particles) and an outer coating formed from a matrix comprising cationic (e.g., high viscosity chitosan) and anionic polymers. The matrix of the outer coating is formed by ionic or other non-covalent interactions, rather than by chemical crosslinking of these polymers.

In certain embodiments, the invention provides a composition useful for delivery of irritating active agents. Such compositions comprise particles having an inner core containing the irritating active agent and an outer coating formed from a matrix comprising a cationic polymer (e.g., high viscosity chitosan biopolymer) and an anionic polymer. In certain embodiments, the active agent is one which is both irritating and water insoluble or slightly water soluble.

In certain such embodiments, the particles described above include inner cores where particles of an active agent are enclosed within droplets of a dispersant (e.g., soybean oil), which are in turn encapsulated in a matrix outer coating comprising a cationic polymer and an anionic polymer.

In an exemplary embodiment, compositions of the invention are formed from an emulsion of active agent particles (e.g., in a suitable dispersing agent) and an aqueous solution of a cationic polymer precipitated under vigorous stirring conditions in the presence of an anionic polymer, for example, at pH values from 5.0 to 6.0 or greater than 6.0, to form microparticles and/or nanoparticles. The particle size (e.g., of active agents) can be reduced, for example, through the use of a high pressure homogenizer (e.g., microfluidizer). Two or more passes through the high pressure homogenizer can be used to obtain particles of the desired size.

In another exemplary embodiment, compositions of the invention are prepared by dissolving a cationic polymer in an aqueous solution and mixing that with an active agent (e.g., retinoic acid) in a suitable dispersing agent to form an emulsion containing active agent particles, which is then directly passed through a high pressure homogenizer until particles of a desired size are obtained. Other agents may be added to the emulsion (e.g., an anionic polymer), preferably agents that are not irritating to the skin, in order to facilitate formation of particles.

In certain embodiments, formulations of the invention include particles of less than 1 micron in diameter, such as less than 500 nm, and preferably greater than 20 nm. Such particles are generally small enough to cross the stratum corneum but large enough to be retained in skin tissue.

The present invention also includes the use of the compositions described herein in the manufacture of a medicament for treating a disease or condition disclosed herein.

The topical delivery of water insoluble active agents in the form of a particulate suspension allows greater stability of the active ingredient.

### BRIEF DESCRIPTION OF THE DRAWINGS

FIG. 1 shows a schematic diagram of an exemplary chitosan gel of the invention.

FIG. 2 shows that retinoic acid is stable at 40° C. in a composition of the invention.

FIG. 3 shows retinoic acid permeation through a skin explant model using Franz Diffusions Cells for free retinoic acid or chitosan-entrapped retinoic acid as a function of the concentration of high molecular weight chitosan (HMW).

FIG. 4 shows the skin distribution of retinoic acid (ATRA) after 200 hours.

### DESCRIPTION OF THE INVENTION

It was found, unexpectedly, that if a cationic polymer, such as high viscosity chitosan, is first mixed in the presence of a water insoluble active ingredient dispersed in a suitable dispersing agent (e.g., as particles) to form a matrix, this matrix can then be precipitated under vigorous stirring conditions in the presence of anionic polymers to form micron-sized gel particles that act as controlled release topical systems (e.g., by controlling penetration of the stratum corneum or outer skin layer). This preparation of particles of an active agent encapsulated by a cationic polymer (e.g., chitosan) avoids the use of surfactants or emulsifiers which can cause skin irritation or other adverse reactions.

The present invention provides compositions where water insoluble or slightly soluble active agents, e.g., pharmaceuticals, such as retinoids, are incorporated into polymeric carriers to provide advantages, such as preferable tissue distribution of the drug, prolonged half life, controlled drug release and reduction of drug toxicity. In addition, typical compositions of the invention provide sustained release of the active agent. While Applicant does not wish to be bound by any particular theory, it is believed that sustained release is obtained by entrapping or precipitating the active agent (and typically the dispersant) in a matrix of cationic and anionic polymers. Furthermore, typical compositions of the invention serve as topical delivery vehicles that do not leave polymeric residues on the skin. The absence of residues may be due to the bioadhesiveness of certain cationic polymers to the skin surface, which is believed to allow for greater penetration into the stratum corneum or the outer layer of the skin. Consistent with the ability of delivery vehicles to reduce skin irritation, exemplified compositions of the invention show statistically lower levels of both erythema and edema in animal studies (see the examples). In certain embodiments, the invention exploits a coacervation complex formed between chitosan and an anionic polymer to encapsulate the chitosan matrix containing the dispersant oil droplets and the active ingredient particles.

The ability to use chitosan, an example of a cationic polymer, in topical pharmaceutical or cosmetic formulations was unexpected. In previous experiments, Applicant found that chitosan was incompatible with anionic polymers and/or a pH greater than 6. Under these conditions, the chitosan precipitates in the form of a gel complex that typically includes cosmetically unacceptable, relatively large particulates in the final topical formula. This is consistent with literature indicating that chitosan will form insoluble precipitates in the presence of anionic polymers and at a pH greater than 6 (see Cognis Company Literature on Hydagen® CMF and Amerchol Company literature on Kytamer™ PC).

The advantages described above can be further enhanced through by using nanoparticles, which are smaller than microparticles. Microparticles have a mean diameter of 1 micron to 100 microns, such as from 1 micron to 50 microns, 1 micron to 20 microns or 1 micron to 10 microns. Typically, nanoparticles have a mean diameter of less than 1 micron or less than 500 nm, such as from 20 nm to 500 nm, from 20 nm to 300 nm, from 50 nm to 200 nm or from 50 nm to 150 nm. Preferably, greater than 90%, greater than 95%, greater than 97%, greater than 98% or greater than 99% of the particles fall within one of these ranges. Preferably, particle uniformity is such that particles in a group having a particular mean diameter are have individual diameters that are within 50% of the mean diameter, such as within 25% or even within 10%.

As used herein, the term “active agent” refers to any substance that when introduced into the body has an effect on either the appearance of tissue to which it is applied, or alters the way the body functions.

The term “water insoluble” refers to any active agent insoluble in water or slightly water soluble. A compound that is slightly soluble has a solubility of less than 0.1 mg/ml and preferably less than 0.05 mg/ml in water at 25° C. A compound that is water insoluble has a solubility of less than 0.01 mg/ml in water at 25° C.

The term “irritating” refers to an active agent that causes edema and/or erythema when applied to skin. Typically, an irritating active agent has a cumulative irritation index (described below) of greater than 1.0, more typically greater than 2.0.

The term “pharmaceutical active” refers to a drug, i.e., a substance which when applied to, or introduced into the body, alters in some way body functions, e.g., altering cell processes. Examples of water insoluble or slightly water soluble pharmaceutical actives include, but are not limited to anti-inflammatory agents (e.g., NSAIDS, hormones and autacoids such as corticosteroids), anti-acne agents (e.g., retinoids), anti-wrinkle agents, anti-scarring agents, anti-psoriatic agents, anti-proliferative agents (e.g., anti-eczema agents), anti-fungal agents, anti-viral agents, anti-septic agents (e.g., antibacterials), local anaesthetics, anti-migraine agents, keratolytic agents, hair growth stimulants, hair growth inhibitors, and other agents used for the treatment of skin diseases or conditions. Certain active agents belong to more than one category.

Examples of retinoids include, but are not limited to, compounds such as retinoic acid (both cis and trans), retinol, adapalene, vitamin A and tazarotene. Retinoids are useful in treating acne, psoriasis, rosacea, wrinkles and skin cancers and cancer precursors such as melanoma and actinic keratosis.

Non-steroidal anti-inflammatory agents include salicylic acid, salicylate esters, acetylsalicylic acid, diflunisal, phenylbutazone, oxyphenbutazone, ibuprofen, ketoprofen, naproxen, mefenamic acid, floctafenine, tolmetin, zomepirac, diclofenac, piroxicam, and the like.

Autacoids and hormones (not limited to anti-inflammatory agents) include steroids, prostaglandins, prostacyclin, thromboxanes, leukotrienes, angiotensins (captopril), as well as pharmaceutically active peptides such as serotonin, endorphins, vasopressin, oxytocin, and the like. Slightly water soluble steroids include estrogen and corticosteroids. Anti-inflammatory corticosteroids include progesterone, hydrocortisone, prednisone, fludrocortisone, triamcinolone, dexamethasone, betamethasone, fluocinolone, and the like.

Local anaesthetics inlcude cocaine, benzocaine, tetracaine, lidocaine, bupivacaine, their hydrochloride salts, and the like.

General antiseptic agents include acridine dyes, bronopol, chlorhexidine, phenols, hexachlorophene, organic mercurials, organic peroxides (benzoyl peroxide), quaternary ammonium compounds, and the like.

Antibiotic agents include penicillins, cephalosporins, cyclosporin, vancomycin, bacitracin, cycloserine, polymyxins, colistin, nystatin, amphotericin B, mupirocim, tetracyclines, chloramphenicol, erythromycin, neomycin, streptomycin, kanamycin, gentamicin, tobramycin, amikacin, netilmicin, spectinomycin, clindamycin, rifampin, nalidixic acid, flucytosine, griseofulvin, and the like. Sulfanilamide antibacterial agents include sulfanilamide, sulfacetamide, sulfadiazine, sulfisoxazole, sulfamethoxazole, trimethoprim, pyrimethamine, and the like.

Antiviral agents include vidarabine, acyclovir, ribavirin, amantadine hydrochloride, rimantadine, idoxyuridine, interferons, and the like.

Anti-fungal agents include miconazole, ketoconazole, terbinafine, tolnaftate, undecylic acid, and other heterocyclic compounds including morpholine, imidazoles and derivatives thereof.

Keratolytic agents include benzoyl peroxide, alpha hydroxyacids, fruit acids, glycolic acid, salicylic acid, ethylhexyl 4-hydroxybenzoic acid, phenyl 4-hydroxybenzoic acid, azelaic acid, trichloroacetic acid, lactic acid and piroctone.

Anti-migraine agents include triptans such as sumatriptan.

Anti-alopecia (hair growth) agents include niacin, nicotinate esters and salts, and minoxidil.

Compounds particlarly useful in treating acne include azelaic acid (an aliphatic diacid with antiacne properties), anthralin (a diphenolic compound with antifungal and antipsoriatic properties), and masoprocol (nordihydroguaiaretic acid, a tetraphenolic compound with antioxidant properties, also useful in the treatment of actinic keratosis) and analogs thereof (such as austrobailignan 6, oxoaustrobailignan 6,4′-O-methyl-7,7′-dioxoaustrobailignan 6, macelignan, demethyldihydroguaiaretic acid, 3,3′,4-trihydroxy-4′-methoxylignan, Saururenin, 4-hydroxy-3,3′,4′-trimethoxylignan, and isoanwulignan).

Active agents particularly effective against proliferative diseases (e.g., cancer, psoriasis) include a residue of alitretinoin (9-cis-retinoic acid); amifostine; bexarotene (4-[1-(5,6,7,8-tetrahydro-3,5,5,8,8-pentamethyl-2-naphthalenyl) ethenyl] benzoic acid); bleomycin; capecitabine (5′-deoxy-5-fluoro-cytidine); chlorambucil; bleomycin; BCNU; cladribine; cytarabine; daunorubicin; docetaxel; doxorubicin; epirubicin; estramustine; etoposide; exemestane (6-methylenandrosta-1,4-diene-3,17-dione); fludarabine; 5-fluorouracil; gemcitabine; hydroxyurea; idarubicin; irinotecan; melphalan; methotrexate; mitoxantrone; paclitaxel; pentostatin; streptozocin; temozolamide; teniposide; tomudex; topotecan; valrubicin (N-trifluoroacetyladriamycin-14-valerate); and vinorelbine. Antimetabolite active agents suitable as one or more constituent compounds in the present invention include: 5-fluorouracil, methotrexate, 5-fluoro-2′-deoxyuridine (FUDR), Ara-C (cytarabine), gemcitabine, mercaptopurine, and other modified nucleotides and nucleosides. Antimetabolite compounds interfere with the normal metabolic processes within cells, e.g., by combining with the enzymes responsible for them, and are generally useful in treating proliferative disorders.

Anti-eczema agents include pimecrolimus and tacrolimus.

Antipsoriatic active agents suitable for use in the present invention include retinoids (including isomers and derivatives of retinoic acid, as well as other compounds that bind to the retinoic acid receptor, such as retinoic acid, acitretin, 13-cis-retinoic acid (isotretinoin), 9-cis-retinoic acid, tocopheryl-retinoate (tocopherol ester of retinoic acid (trans- or cis-)), etretinate, motretinide, 1-(1 3-cis-retinoyloxy)-2-propanone, 1-(13-cis-retinoyloxy)-3-decanoyloxy-2-propanone, 1,3-bis-(13-cis-retinoyloxy)-2-propanone, 2-(13-cis-retinoyloxy)-acetophenone, 13-cis-retinoyloxymethyl-2,2-dimethyl propanoate, 2-(13-cis-retinoyloxy)-n-methyl-acetamide, 1-(13-cis-retinoyloxy)-3-hydroxy-2-propanone, 1-(13-cis-retinoyloxy)-2,3-dioleoylpropanone, succinimdyl 13-cis-retinoate, adapalene, and tazarotene), salicylic acid (monoammonium salt), anthralin, 6-azauridine, vitamin D derivatives (including but not limited to Rocaltrol (Roche Laboratories), EB 1089 (24α,26α,27α-trihomo-22,24-diene-1α,25-(OH)₂-D₃), KH 1060 (20-epi-22-oxa-24α,26α,27α-trihomo-1α,25-(OH)₂-D₃), MC 1288, GS 1558, CB 1093, 1,25-(OH)₂-16-ene-D₃, 1,25-(OH)₂-16-ene-23-yne-D₃, and 25-(OH)2-16-ene-23-yne-D₃, 22-oxacalcitriol; 1α-(OH)D₅ (University of Illinois), ZK 161422 and ZK 157202 (Institute of Medical Chemistry-Schering AG), alfacalcidol, calcifediol, calcipotriol (calcipotriene), maxacalcitriol, colecalciferol, doxercalciferol, ergocalciferol, falecalcitriol, lexacalcitol, maxacalcitol, paricalcitol, secalciferol, seocalcitol, tacalcitol, calcipotriene, calcitriol, and other analogs as disclosed in U.S. Pat. No. 5,994,332), pyrogallol, and tacalcitol.

Additional pharmaceutical actives for skin diseases include antihistamines, capsaicin, resiquimod and imiquimod. Further pharmaceutical actives include antigens such as proteins (including glycoproteins and lipoproteins) such as tetanus toxoid and diphtheria toxoid, carbohydrates, viral particles and whole attenuated or deactivated viruses (e.g., influenza virus).

The term “therapeutic active” as used herein, refers to an insoluble or a slightly water soluble substance which either alters processes within the body, or alters the cosmetic appearance of the tissue of interest, e.g., skin, but is not technically considered a drug (pharmaceutical active agent). Examples of therapeutic active agents include, but are not limited to, vitamins and vitamin derivatives, skin coloring and bleaching agents (e.g., dihydroxyacetone), skin protectants, moisturizers, depilatories, soap and other cleansers, emollients, moisturizers and peels.

Vitamins and derivatives thereof include Vitamin A, ascorbic acid (Vitamin C), alpha-tocopherol (Vitamin E), 7-dehydrocholesterol (Vitamin D), Vitamin K, alpha-lipoic acid, lipid soluble anti-oxidants, and the like.

Exemplary skin protectants suitable as an active agent in the present invention include allantoin and esculin.

Depigmenting agents include hydroquinone, 2,5-dihydroxybenzoic acid and kojic acid.

Other therapeutic active agents include seabuckthorn oil and aromatic oils such as orange oil.

The term “chromogenic active”, as used herein refers to water insoluble or slightly water soluble sunscreens. Examples of sunscreens are octylmethoxycinnamate and related esters, octyl salicylate and esters, para-aminobenzoic acid and esters, benzophenones such as 2-hydroxy-4-methoxybenzophenone, benzyldiphenyl acrylates, anthranilates, triazines, benzylidenecamphor and derivatives. Further exemplary sunscreens suitable as an active agent in the present invention include actinoquinol, p- and 4-dimethylaminobenzoic acid.

In certain embodiments, the composition contains more than one active agent, i.e., comprises at least one additional active agent, which can be either a pharmaceutical active, chromogenic active or a therapeutic active. For example, a composition includes a retinoid as a pharmaceutical active and vitamin E as a therapeutic active.

The invention will be primarily discussed in relation to retinoids. However, it is to be understood that any active agent that can be used in a delivery system can be used in the compositions and methods of the present invention. Preferably, the active agent is a water insoluble substance. Exemplary agents include retinoids, e.g., retinoic acid and retinol (Vitamin A), calcipotriene, and other active agents which are known to cause irritation of the skin.

The term “topical” as used herein is known in that art and includes the application of the compounds of the present invention to epithelial surfaces, including skin, mucosal membranes of the nasal and upper respiratory system, digestive and gastrointestinal tract.

The term “cationic polymer” as used herein includes a component of the delivery system that assists in the release of the active agent that is being delivered. A preferred cationic polymer is a high viscosity chitosan having a molecular weight of at least about 30,000, such as at least about 100,000 Daltons, more preferably at least about 250,000 Daltons and most preferably at least about 300,000 Daltons. In one example, cationic polymers suitable for use in the invention have one positive charge (or a moiety capable of being positively charged when applied to the skin) per 100 amu to 2000 amu. Examples of such polymers include albumin, gelatin, starch, DEAE-Cellulose, cationic guar and DEAE-Dextran. DEAE-Dextran and cationic guar have tertiary amino groups. Cationic guar's INCI name is Guar hydroxypropyltrimonium chloride and DEAE-Dextran is Diethylaminoethyl-Dextran. Additional examples of such cationic polymers are those having one or more hydrophobic regions, disclosed in U.S. Pat. Nos. 6,264,937, 6,299,868 and 6,726,906, the contents of which are incorporated herein by reference.

Suitable cationic polymers, such as chitosan and the polymers disclosed in the cited patents, often have a high capacity for binding lipids. For example, the capacity of chitosan for lipids is 5380 relative units, as compared to other biodegradable polysaccharides such as methylcellulose (lipid capacity of 128) when tested in an oral fat uptake in vivo assay (Watanabe et al., 1992).

Cationic polymers are preferably not covalently crosslinked, such as with glutaraldehyde or a divalent crosslinking agent. In addition, cationic polymers used in the invention are preferably biodegradable.

Chitosan is a natural, biodegradable cationic polysaccharide derived by deacetylating chitin, a natural material extracted from fungi, the exoskeletons of shellfish and from algae and has previously been described as a promoter of wound healing (Balassa, U.S. Pat. No. 3,632,754 (1972); Balassa, U.S. Pat. No. 3,911,116 (1975)). Chitosan comprises a family of polymers with a high percentage of glucosamine (typically 70-99%) and N-acetylated glucosamine (typically 1-30%) forming a linear saccharide chain of molecular weight from 10,000 up to about 1,000,000 Dalton. Typically, chitosan used in the invention is 70-100% glucosamine, such as 70-90% glucosamine or 80-100% glucosamine, more typically 85-95% glucosamine. Chitosan, through its cationic glucosamine groups, interacts with anionic proteins such as keratin in the skin conferring some bioadhesive characteristics. In addition, chitosan has also a high affinity for lipids and fats, which, for example, allows targeting to the hair follicle based on chitosan's high affinity for sebum fluids. In addition, when not deacetylated, the acetamino groups of chitosan are a target for hydrophobic interactions and contribute to some degree to its bioadhesive characteristics (Muzzarelli et al., In: Chitin and Chitinases, Jolles P and Muzzarelli RAA (eds), Birkhauser Verlag Publ., Basel, Switzerland, pp. 251-264 (1999)). Chitosan is also believed to prevent the reagglomeration of active particulates.

A cationic polymer can be selected to obtain a polymer having a desired elasticity. In certain embodiments, polymers of the invention are selected to have a relatively high elasticity. Elasticity of chitosan is believed to increase with molecular weight. Other cationic polymers can be selected to have an elasticity equal to or greater than chitosan of one of the molecular weights or molecular weight ranges described herein.

The term “high viscosity” chitosan refers to a chitosan biopolymer having an apparent viscosity of at least about 100 cps for 1% solutions in 1% acetic acid as measured using a Brookfield LVT viscometer at 25° C. with appropriate spindle at 30 rpm. The viscosity of the chitosan solution can readily be determined by one of ordinary skill in the art, e.g., by the methods described in Li et al., Rheological Properties of aqueous suspensions of chitin crystallites. J Colloid Interface Sc 183:365-373, 1996. In addition, viscosity can be estimated according to Philipof's equation: V=(1+KC)⁸, where V is the viscosity in cps, K is a constant, C is the concentration expressed as a fraction (Form No. 198-1029-997GW, Dow Chemical Company). In certain embodiments, the high viscosity chitosan preferably has a viscosity greater than at least 100 cps, and more preferably greater than at least 500 cps. In general, the release of an active agent from a composition of the invention is slowed by increasing the viscosity of the cationic polymer, either by increasing the concentration or increasing the molecular weight.

The desired viscosity of the chitosans can be achieved by manipulating the concentration, i.e., percentage and/or molecular weight of chitosans, as shown in the table below, where LMW is chitosan having a molecular weight of less than 50 kDa, MMW is chitosan having a molecular weight of 50-250 kDa and HMW is chitosan having molecular weight greater than 250 kDa:

| | | |
|---|---|---|
| LMW | MMW | HMW |

| Viscosity | | Viscosity | | Viscosity | |
|---|---|---|---|---|---|
| (cps) | % | (cps) | % | (cps) | % |
| 7 | 1 | 66 | 1 | 552 | 1 |
| 21,263 | 9 | 151,403 | 5 | 15,862 | 2 |
| 116,882 | 12 | 3.27E+06 | 8 | 171,163 | 3 |

In certain embodiments, the chitosan has a molecular weight of at least 300,000 Daltons (e.g., 300 kDa to 1,000 kDa, 500 kDa to 1,000 kDa). In other preferred embodiments, the chitosan has a concentration of at least 1 weight %, typically at least about 2 weight %. In an especially preferred embodiment, the biopolymer comprises a high viscosity chitosan having a molecular weight of at least about 300,000 Daltons (e.g., 300 kDa to 1,000 kDa, 500 kDa to 1,000 kDa) and at a concentration of at least 2 weight %.

The term “dispersing agent” as used herein comprises any suitable agent that will suspend the water insoluble or slightly water soluble active agent but does not chemically react with either chitosan or the active substance. Preferably, the active agent is compatible with the dispersing agent, but is not freely soluble in the dispersing agent such that a fraction, preferably at least 70% such as at least 80% or 90%, of the particles is not dissolved. For example, the active agent is typically about 0.001% to about 10% (e.g., about 1% to about 10%) soluble in the dispersing agent under the conditions used to make the microparticles or nanoparticles. In other words, the active agent generally has a solubility of less than 10 mg/mL, such as less than 1 mg/mL, less than 0.1 mg/mL or less than 0.001 mg/mL in the dispersant at 25° C. Suitable dispersing agents typically have a polarity index values (where water has a polarity index of 9) 0.5 to 5 units less than the solvents in which an active agent is freely soluble. Examples of dispersing agents, particularly for retinoic acid and other active agents having similar solubility characteristics, include squalane, triglycerides, (e.g., medium chain, miglyol-812, caprylic, 4-capric, 4-stearic, soybean oil (including partially hydrogenated variations), coconut oil, almond oil, olive oil, safflower oil, cotton seed oil), cholesterol and cholesterol esters (e.g., cholesterol oleate), Softifan 378, silicone oil, mineral oil, dibutyl hexanedioate, oleic acid, palmitic acid, palmitoleic acid, wax esters (paraffin, spermaceti) yellow wax, cocoglycerides, lipid components of sebum, aliphatic or aromatic esters having 2-30 carbon atoms (e.g., cococaprylate/caprate), alkyl, aryl, or cyclic ethers having 2-30 carbon atoms (e.g., butyl ether, isopropyl ether), linear, cycloaliphatic or aromatic hydrocarbons having 4-30 carbon atoms, alkyl or aryl halides having 1-30 carbon atoms. In general, a greater proportion (e.g., more than 50%, 60%, 70%, 80% or typically more than 90%) of active agent particles partition into a suitable dispersing agent than into aqueous solution.

Antioxidants and UV filters (e.g., D,L-tocopherol, BHT, BHA) may be added to the dispersant to protect the active particles from undergoing oxidative or UV-induced degradation.

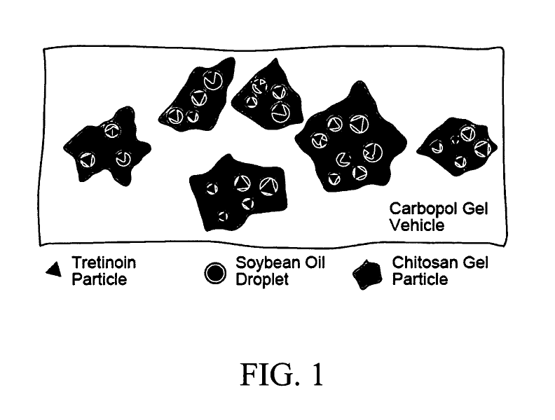

In embodiments where the active agent particles are enclosed within dispersant oil droplets, the oil droplets are in turn enclosed by the chitosan gel phase as shown in FIG. 1. The optimal size of the dispersed oil droplets is typically a function of the active particle size, such that size of the oil droplet increases as the particle size increases. Typically, active agent particles of the invention have a diameter of 0.1-10 microns, such as 0.1-1 (e.g., for sunscreen or cancer chemotherapeutics) or 1-10 (e.g., for anti-acne agents or agents where the hair follicles are targeted) microns, particularly 5-10 microns (e.g., as measured by light scattering with, for example, a Horiba 300 series instrument). Typically, oil droplets of the invention have a diameter of 1-100 microns, such as 10-70 microns, particularly 30-50 microns. In certain embodiments, the size of active agent particles, such as after one or more passes through a homogenizer (microfluidizer), is measured prior to addition of chitosan or another cationic polymer. In this step, both particle size and uniformity can be verified.

In general, enough chitosan matrix is present to coat the oil droplets, thereby increasing the total gel particle size by 0.1 to 100 microns (for a typical total particle diameter of 30-150 microns). Exemplary gel particles have a number average and/or volume average diameter of less than 100 microns, such as less than 50 microns.

Control of the dispersant droplet size and uniformity within the chitosan gel phase is generally accomplished using a homogenizer. The encapsulating chitosan gel acts as a controlled release vehicle, regulating the permeation of active agent out of the oil droplets and into the upper layers of the skin. Functionally, the chitosan gel particles in the gel vehicle (e.g. polyacrylic acid) should be small enough for the final gel to appear smooth and uniform to the eye, but large enough that the oil droplets and active particles are located within the interior of the chitosan gel particles, and not at the interface between the chitosan gel and the gel vehicle. While Applicant does not wish to be bound by theory, it is believed that the release of active is controlled by the diffusion path length between the active/dispersant droplets and the surface of the chitosan gel.

After the high pressure homogenization these cationic biopolymers can complex with anionic polymers such as polyacrylate (carbomer) gels or other types of anionic gels to further stabilize the biopolymer-entrapped drug particles. However, the anionic polymer content preferably does not result in a gel particle having a neutral or negative charge, such that there are a greater number of positive charges than negative charges in a gel particle. Typically, the ratio of positive to negative charges is 1:1 to 5:1, such as 1.5:1 to 4:1 or 1.5:1 to 2.5:1.

Alternatively, the desired ratio of cationic polymer to anionic polymer can be determined by measuring the viscosity of the composition. Typically, the viscosity of the composition is at least 10 times, at least 20 times, at least 50 times, or at least 100 times greater following addition of the anionic polymer.

The term “anionic polymer” refers to negatively charged polymers which can form a complex with a cationic polymer such as chitosan. Anionic polymers generally have groups such as carboxylate, phosphonate, phosphate, and sulfonate attached directly or indirectly to a backbone or part of a backbone such as a polysaccharide, a polyacrylate or a polyethylene. Examples of anionic polymers include poly(acrylic acid) and derivatives, xanthan gum, alginates (e.g., sodium alginate), gum arabic, carboxy methylcellulose, hydroxypropyl methylcellulose, ethylcellulose, carrageenan, polyvinyl alcohol, sulfated glycosaminoglycans such as chondroitin sulfate and dermatan sulfate. The molecular weight of an anionic polymer can be selected by one of ordinary skill in the art, but is generally from 50,000-1,000,000 Daltons. Typically, the viscosity of a 1% solution of an anionic polymer is from 50,000-100,000 cps. Anionic polymers used in the invention may create non-covalent crosslinking between the cationic polymers.

To produce particles according to the invention, one embodiment includes forming a matrix of a viscous aqueous solution of a cationic polymer (e.g., chitosan) and a water insoluble active agent or an oil component containing a water insoluble active agent by vigorous stirring (e.g., stirring that creates sufficient shearing to produce particles of 100 microns or less in mean diameter, such as that generated by a homogenizer, e.g., a Y-type homogenizer) in a first step. For chitosan, the molecular weight is preferably more than 30,000 or 100,000 Daltons and at a concentration greater than 2 wt %. The matrix is then precipitated by adding an anionic polymer solution under vigorous stirring, e.g., at a pH from 4-6, 5-6, or greater than 6, such as pH 6-8, which results in the formation of microparticles. The viscosity of the precipitated particles is typically at least 50,000 cps, such as at least 100,000 cps. The size of the partcles can be reduced using a high pressure homogenizer.

In another embodiment, particles are prepared using a high pressure homogenizer such as a Microfluidizer (Model M-110Y; Microfludics Corporation, Newton, Mass.) which reaches pressures up to 20,000 psi. Other homogenizers capable of pushing a suspension through fine channels, mesh or screening at high pressure (e.g., pressure of 3000 psi or greater, such as at least 5000 psi or at least 10000 psi) thereby generating shearing force capable of reducing particle size, such as a French press, are also suitable. With a high pressure homogenizer, it is possible to create an emulsion of the solid active ingredient suspended in a mixture of lipids in an aqueous cationic polymer solution. Two or more passes through the homogenizer may be required to achieve the desired particle size. The polymer/lipid emulsion forms layers around the microscopic drug particles, thereby forming a stable suspension. An anionic polymer is added to the suspension to precipitate the matrix. Particle size reduction following addition of the anionic polymer can be achieved by passing the precipitated matrix through a homogenizer (e.g., a Z-type homogenizer).

All or part of microparticle or nanoparticle preparation is advantageously conducted under an inert atmosphere, particularly steps prior to precipitation with an anionic polymer. Typically, the inert atmosphere consists of one or more of nitrogen, helium, argon and other inert gases. Hydrogen can also be present when the active ingredient is sensitive to oxidation but not reduction. For example, a microfluidizer can be maintained under a nitrogen atmosphere.

Separately or in combination with the inert atmosphere described above, all or part of microparticle or nanoparticle preparation is optionally conducted in the absence of light. If not all light can be excluded, light of longer wavelengths (e.g., red light) is preferred to minimize risk of photodamage to a compound. Exclusion of light is desirable for photosensitive compounds, particularly the retinoids.

In certain embodiments, the composition includes a preservative. Typically, the preservative is an antioxidant. Exemplary antioxidants include BHT, BHA, vitamin E and other tocopherols and vitamin C (ascorbic acid). In a particular embodiment, the weight ratio of antioxidant to active agent (e.g., a retinoid such as retinoic acid) is about 1:3 to 3:1, such as about 1:2 to 2:1, for example, 1.5:1 to 1:1.5.

Following formation of microparticles and/or nanoparticles stabilized by an anionic polymer, the composition containing the microparticles and/or nanoparticles is typically added to a suitable vehicle to prepare a pharmaceutical formulation (e.g., a gel, cream or lotion). Preferably, the vehicle does not disrupt the microparticles or nanoparticles and instead stabilizes the particles. An example of preferred components of a vehicle is a polymeric viscosity enhancer such as hydroxyethylcellulose and/or a chelator such as EDTA. A vehicle also advantageously includes preservatives such as antioxidants and/or antimicrobials. In addition, the vehicle preferably does not irritate or otherwise damage the tissue to which it is administered. For example, vehicles for topical formulations typically exclude ethanol, isopropanol, emulsifiers and surfactants.

Pharmaceutical formulations of the invention typically have a viscosity of at least 100,000 cps, such as at least 200,000. For example, a pharmaceutical formulation can have a viscosity of 100,000 to 500,000 cps, such as 200,000 to 300,000 cps.

A pharmaceutical formulation can be prepared and/or packaged under an inert atmosphere.

Pharmaceutical formulations of the invention can be administered by various routes, such as topically, transdermally or transmucosally (e.g., intranasal administration, buccal administration). Typically, pharmaceutical formulations of the inventions are administered to a surface, such as the skin or a mucous membrane.

The amount of water insoluble active employed will be that amount necessary to deliver a pharmaceutically or therapeutically effective amount to achieve the desired result at the site of application. In practice, this will vary depending upon the particular medicament, severity of the condition and other factors. In general, the concentration of the actives in the final formula can vary from as little as 0.0001 up to 20 percent or higher, by weight of the final formula. For retinoids, a preferred dose is between 0.01%-1% for retinol and between 0.01%-0.1% for all-trans-retinoic acid. Typically, the amount of water insoluble active corresponds to no more than 10% by weight of a microparticle or nanoparticle composition in the final formulation.

Diseases and conditions that can be treated with compositions of the invention include acne, psoriasis, seborrheic dermatitis, aging and photoaging (photodamage) of the skin, wrinkles, actinic keratosis, melanoma, hair growth disorders (e.g., baldness, hirsutism), warts, dry and/or scaly skin and rosacea.

The relative irritation caused by a composition can be assessed through measuring the “irritation index”, which is analogous to a therapeutic index. The irritation index is the ratio of irritation to efficacy. Methods of measuring the irritation index are known in the art and are described below in Example 7, namely the Draize test on New Zealand white rabbits.

Irritation can be measured as erythema on a 5-point scale is plotted on a logarithmic scale versus the concentration of a compound. The 5-point scale is as follows:

| Level | Irritation |
|---|---|
| 0 | No erythema |
| 1 | Very slight erythema |
| 2 | Well defined erythema |
| 3 | Moderate to severe erythema |
| 4 | Severe erythema |

Efficacy, such as for acne, is assessed by measuring the reduction in the size of acne lesions on a logarithmic plot versus concentration.

Prior art preparations typically have an irritation index of about 1 to 4. Preparations of the invention using nanoparticles generally have an irritation index of greater than 10, such as from 10 to 20 or 10 to 15.

Irritation can also be measured by patch testing assay (see Cattaneo and Demierre, Drug Del. Technol. 1:45 (2001) and Queille-Roussel et al., Clin Ther. 23(2):205-12 (2003)), the contents of which are incorporated herein by reference). This patch testing assay can be used to determine a cumulative irritation index, where white petrolatum serves as a negative control and conventional tretinoin formulations have an index of about 2.0-2.5. Compositions of the invention typically have an index of less than 1.5 and the index is advantageously less than 1.0 or even 0.5 or 0.25. Irritation can also be assessed by the methods described by Fluhr et al., Br. J. Dermatol. 145:696-703 (2001), the contents of which are incorporated herein by reference. These methods include laser-doppler perfusion imaging (LDI), laser-doppler flowmetry (LDF), transepidermal water loss, visual scoring (VS), colorimetrix measurements, the Mexameter hemoglobin scale (Mexa Hb) and capacitance. LDI, LDF, Mexa Hb and VS are particularly useful for determining the extent of irritation caused by retinoic acid.

Toxicity of compositions can be measured by, for example, the MTT assay. Compositions of the invention have at least 90%, such as least 95%, 98% or 99% viability in the MTT assay.

Preparations containing particles are typically taken up readily by the skin, so that absorption of an active agent is increased and ghosting is minimized. In one example, 0.5-5% by weight, such as 0.5-2% or 2-5%, of an active agent is delivered to the skin in the 24 hours after a preparation is applied. Ghosting can be measured by removing the preparation remaining on the skin with adhesive tape and measuring the amount of active agent or another part of a preparation. Typically, a preparation containing particles of the invention will have at least 25%, at least 50% or at least 75% less residue from a preparation on the skin, as compared to a conventional preparation. Preferably, no residue from particles of the invention can be detected by eye and/or measured using the above method one hour after the particles are administered.

As shown below in the examples, active agents in a particle may be stabilized against, for example, oxidation and light damage. For example, an active agent in a particle and/or a formulation for administration preferably has a half-life at 40° C. of at least 2 weeks, 1 month, 2 months, 3 months, 6 months or 1 year. This half-life can be at least 10%, at least 20%, at least 25%, at least 30%, at least 40% or at least 50% greater than the half-life of an active agent not contained in a particle, under the same storage conditions.

Particles of the invention are generally physically stable, such that separation of the particles occurs slowly, even in the presence of shearing forces associated with formulating the particles and administering them in a composition. For example, particles can have less than 50%, less than 75%, less than 80% or less than 90% separation over a 6 month period.

The invention will now be described in greater detail by reference to the following non-limiting examples:

### EXAMPLE 1

Preparation of Retinoic Acid Particles

Water-insoluble all-trans retinoic acid (ATRA) in the form of solid particles (2 wt %) was incorporated into high viscosity chitosan solutions [3 wt % solutions of Protasan UP B 80/500 (FMC Biopolymers Inc.; 755 cps apparent viscosity) in 2.1 wt % glycolic acid and 0.03 wt % sodium hydroxide] in the presence of soybean oil (17 wt %) by vigorous mixing to form a matrix. The viscosity of the matrix was initially 215,000 cps as measured on a Brookfield LVT viscometer at 25° C. with appropriate spindle at 1.5 rpm. The emulsion was then mixed with a poly(acrylic acid) solution (0.5 wt %) at pH 6.3 and homogenized to make a gel containing retinoic acid microparticles of size below 10 microns.

Particles prepared by this method, when measured with a Horiba LA-910 light scattering device, had a median diameter of 98.1 microns, a mean diameter of 108.0 microns, a geometric mean diameter of 76.7 microns and a mode diameter of 109.2 microns. Approximately 2% of the particles had a diameter of one micron or less.

### EXAMPLE 2

Stability of Retinoic Acid Particles

The concentration of retinoic acid in the final gel formulation was measured by HPLC. Fifty microliters of the topical preparation containing retinoic acid was shaken for 20 minutes in the presence of 5 milliliters of acetonitrile then centrifuged at 4000 rpm for 5 minutes. A 20 microliter aliquot of the supernatant was then injected onto a Zorbax SB-C18 column (4.6 mm×75 mm, 3.5 micron) equipped with a Zorbax SB-C18 Guard cartridge (4.6×12.5 mm) and operated with aq. 70% acetonitrile containing 5% acetic acid and 0.02% triethanolamine as mobile phase (1 ml/mn) and detection at 340 nm. The calibration was linear from 50 to 5,000 ng/ml.

The stability of the retinoic acid was determined over a 3 month period. The retinoic acid was highly stable in the chitosan microparticulates. The initial retinoic acid concentration was determined as 0.052% at time 0 and 0.05% at 3 months.

### EXAMPLE 3

Preclinical Study Involving Gel Formulation Containing Retinoic Acid Particles

A 3-month preclinical study was undertaken in both mice and rabbits to determine the severity of skin reactions after application of the retinoic acid gel as described above using the Draize test. The animals (40 New Zealand White Rabbits and 140 CD-1 mice) were divided into 5 groups as shown in Table 1, 2 and 3.

The test compound was formulated to include a concentration of 0.05 wt % of retinoic acid in microparticulate form as illustrated in Example 1 and applied at 100 times and 500 times the human dose (Groups 3 and 4). The vehicle gel and the vehicle gel containing the chitosan microparticles without retinoic acid (Groups 1 and 2) acted as negative controls whereas a commercial 0.05% cream (Renova 0.05% retinoic acid) in a standard emulsion formula at 500 the human dose (Group 5) acted as positive control. As shown in Table 1 in the rabbit study, it was soon apparent that the positive control was too irritating for the animals and three steps were taken to manage the toxicity of the positive control group: (1) the positive control dose was scaled back to 100 times the human dose from 500 the human dose after 10 days of application; (2) a second site of application of the positive control was required while waiting for the first site to heal (about 2 weeks later) and (3) the animals which displayed the greatest discomfort were given an intramuscular injection of buprenorphine (2 out of 8 animals). As shown in Table 1, the microparticulate delivery system alone did not cause erythema or edema and treatment groups 3 and 4 showed a statistically significant lower irritation and edema level compared to group 5.

TABLE 1
Rabbit study -Average Erythema and Edema
Scores at 10 days post treatment

| | | Average | Average | |
|---|---|---|---|---|
| | | Erythema | Edema | |
| Group Number | | (n = 8) | (n = 8) | Comment |
| 1. | Vehicle | 0.125 | 0 | No erythema/ |
| | | | | edema |
| 2. | Vehicle + Microparticles | 0 | 0 | No erythema/ |
| | (chitosan delivery system | | | edema |
| | only, no ATRA) | | | |
| 3. | Vehicle + ATRA | 0.125 | 0 | No erythema/ |
| | Microparticles (100 | | | edema |
| | human dose) | | | |
| 4. | Vehicle + ATRA | 0.875 | 0.25 | Very Slight |
| | Microparticles (500 | | | erythema; |
| | human dose) | | | no edema |
| 5. | Renova ® | 2.125 | 1.875 | Well defined |
| | (500 human dose) | | | erythema; |
| | | | | slight edema |

After treating the second application site with Renova® at the lower dose (100 times the human dose) for an additional 14 days the level of erythema and edema significantly exceeded the test compound at 100 and the 500 times the human dose (Table 2).

TABLE 2
Rabbit study -average erythema and edema
scores at 24 days post-treatment

| | | Average | Average | |
|---|---|---|---|---|
| | | Erythema | Edema | |
| Group Number | | (n = 8) | (n = 8) | Comment |
| 1. | Vehicle | 0.125 | 0 | No erythema/ |
| | | | | edema |
| 2. | Vehicle + Microparticles | 0 | 0 | No erythema/ |
| | | | | edema |
| 3. | Vehicle + ATRA | 0 | 0 | No erythema/ |
| | Microparticles (100 | | | edema |
| | times the human dose) | | | |
| 4. | Vehicle + ATRA | 1.75 | 0.75 | Slight erythema; |
| | Microparticles (500 | | | very slight edema |
| | times the human dose) | | | |
| 5. | Renova ® (100 times | 2.125 | 1.5 | Well defined |
| | the human dose)* | | | erythema; slight |
| | | | | edema |

*Applied on second site starting at day 10

In the mice study, at 10 days post treatment the positive control group (Group 5-Renova®) receiving 100 times the human dose had significantly more erythema than Group 4 treated with 500 times the human dose of test compound (Table 3):

TABLE 3
Mice Study -Average Erythema and Edema
Scores at 10 days post-treatment

| | | Average | Average | |
|---|---|---|---|---|
| | | Erythema | Edema | |
| Group Number | | (n = 28) | (n = 28) | Comment |
| 1. | Vehicle | 0 | 0 | No erythema/ |
| | | | | edema |
| 2. | Vehicle + Microparticles | 0.07 | 0 | No erythema/ |
| | | | | edema |
| 3. | Vehicle + ATRA | 0.57 | 0.25 | Very slight |
| | Microparticles (100 | | | erythema; |
| | times the human dose) | | | no edema |
| 4. | Vehicle + ATRA | 1.0 | 0.46 | Very slight |
| | Microparticles (500 | | | erythema; |
| | times the human dose) | | | no edema |
| 5. | Renova ® (100 times | 1.67 | 1.21 | Well defined |
| | the human dose)* | | | erythema; |
| | | | | slight edema |

The preclinical results indicate that the test compound is significantly less irritating than a commercial retinoic acid preparation (Renova® 0.05%) in both rabbit and mice studies. These results strongly suggest the potential for increasing patient compliance in patients undergoing retinoic acid therapy for skin diseases such as acne, photodamage and prevention of melanoma.

### EXAMPLE 4

Preparation of Alpha-Lipoic Acid Particles.

The slightly water soluble substance alpha lipoic acid (1.5 wt %) was mixed with an aqueous high viscosity chitosan solution (8.3 wt %, 40,000 cps) and soybean oil (0.8 wt %) under vigorous stirring. The pH of the emulsion was then raised to 6.3 using triethanolamine under vigorous stirring conditions to precipitate the chitosan matrix (no anionic polymer was used in this procedure). The microparticle size was 5 microns. The emulsion was then passed through a Microfluidizer® to obtain particle sizes approximately 500 nm in diameter after 5 passes through the Microfluidizer® filters. The microfluidizer pushes the emulsion through very fine pore filters at high pressure (greater than 1000 psi) which causes a reduction in particle size.

### EXAMPLE 5

Preparation of Octylmethoxycinnamate Sunscreen Particles.

The slightly water soluble sunscreen octylmethoxycinnamate (7.5 wt %, 40,000 cps) was first mixed with an aqueous high viscosity chitosan solution (10 wt %) to form an oil in water (O/W) emulsion. The emulsion was then mixed with an aqueous xanthan gum solution (40 wt %) to further increase the viscosity of the sunscreen/chitosan (O/W) emulsion. In a separate container microfine zinc oxide (9 wt %) was mixed with an oily solution containing cocoglycerides (12 wt %), lauryl glucoside (3 wt %), polyglyceryl-2-dipolyhydroxystereate (1 wt %) and sodium cetearyl sulfate (1 wt %) heated at 70° C. The sunscreen/chitosan O/W emulsion and the oily solution containing microfine zinc oxide were then mixed together using a high speed mixer. The final pH of the sunscreen was 7.0 which caused precipitation of the chitosan matrix in the form of microparticles containing the sunscreen agent. The mixture was then cooled below 40° C. before adding preservative.

### EXAMPLE 6

Preparation of Particulate Hydrogels

A chitosan hydrogel was prepared by dissolving 3% w/w chitosan with a molecular weight greater than 300 kDa into a water solution containing 2% w/w glycolic acid and 0.3% w/w sodium hydroxide. 6 grams of BHT were dissolved in 50 grams of soybean oil and this solution was added to 250 grams of the hydrogel without stirring. 5 grams of retinoic acid in powder form were added to the oil layer and mixed under moderate conditions with the hydrogel to form a first emulsion. 200 grams of saline (0.9% NaCl) were added to the first emulsion which was then passed through the high pressure homogenizer to reduce the particle size. The reduction in particle size is a function of the number of passes through the high pressure homogenizer (110Y Microfluidizer); two passes were sufficient to achieve the desired size, although more can be used. The resulting product is composed of tretinoin particles with at least 90% of the retinoid particles less than 20 microns in suspensions (particles in a liquid) and/or emulsions (droplets in liquids). The particle size was measured using a Horiba LA 910 particle analyzer which can measure particle sizes down to 20 nm. The biopolymer forms a matrix around the microscopic drug particles and dispersant droplets, which enables them to form a stable suspension and prevents agglomeration. This particulate composition was then further mixed in a standard anionic gel to make the final hydrogel preparation as illustrated below.

| Tretinoin Gel FORMULATION | % |
|---|---|
| Tretinoin | 0.05 |
| Soybean Oil | 0.5 |
| Butylated hydroxytoluene | 0.06 |
| Chitosan (80% deacylated, MW > 500 Da | 0.076 |
| Glycolic Acid | 0.05 |
| Sodium Hydroxide | 0.0076 |
| Sodium Chloride | 0.018 |
| Edetate disodium | 0.095 |
| Carbomer | 0.475 |
| Trolamine | 0.57 |
| Propylene Glycol | 0.56 |
| Imidazolidinyl Urea | 0.3 |
| Methylparaben | 0.11 |
| Propylparaben | 0.03 |
| Purified Water | q.s |

Gel Formulation

| | Ingredient | Wt. % |
|---|---|---|
| A | Deionized water | 92.8 |
| | Disodium EDTA | 0.1 |
| | Carbomer | 0.5 |
| B | Triethanolamine | 0.6 |
| C | Tretinoin particles in soybean/chitosan matrix | 5.0 |
| D | Preservatives | 1.0 |
| | Total | 100.0 |

The Part A ingredients were weighed into a suitable vessel equipped with a mixer. The mixture was mixed at room temperature until uniform. Part B was added to neutralize the gel. The Part C ingredient was separately added under low shear conditions until a homogenous mixture (gel) was formed. Part D was added for the final preparation.

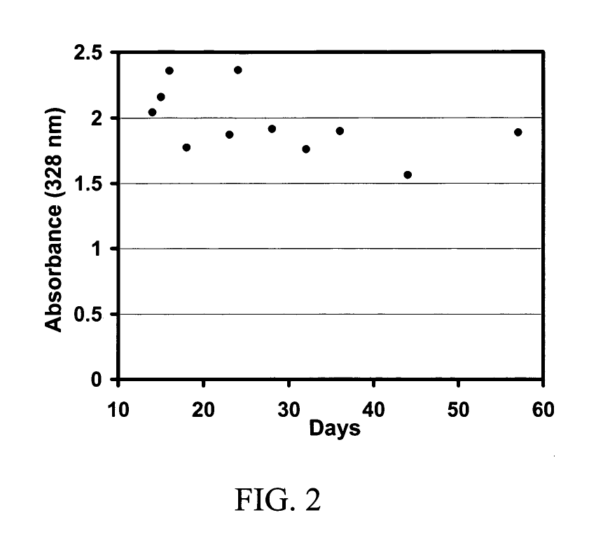

As shown in FIG. 2, retinoic acid entrapped in 3% high molecular weight chitosan (HMW) was highly stable at 40° C. This remedies a historic disadvantage of retinoids, their photochemical instability. Under the influence of light, especially at elevated temperatures, the material is rapidly degraded. Attempts have been made to solve the problem of inadequate stability in a variety of ways. For example, these include: storage of the material under inert conditions, addition of antioxidants such as vitamin E or BHT, and by use of lightproof packs. However, it has been found that only entrapment of retinol in a matrix has proved to be of any practical value. Comparison of different matrix materials has shown that a chitosan matrix is clearly superior to other matrices. When chitosan-based particles are employed as a delivery system for retinoids, skin care preparations also show significantly greater activity than products containing commercially available retinoid delivery systems.

The final active-containing gels were also tested for their capacity to hold retinoic acid after equilibration with a phosphate buffer solution containing a surfactant (0.5% Volpo). The all trans-retinoic acid (ATRA) release was monitored by HPLC (HP1090) and found to be 533 ng/mg for chitosan (90% deacetylated, 360,000 Dalton MW), 426 ng/mg for cationic guar and 183 ng/mg for DEAE-Dextran, respectively, as compared to 19 ng/mg for Gum Arabic.

The role of vehicle on the absorption of topically applied retinoids and the distribution of retinoid within skin was examined using Franz Diffusion Cells which are as described in Lehman Pa., Slattery J T and Franz T J. Percutaneous Absorption of Retinoids: Influence of Vehicle, Light Exposure, and Dose. J Invest Dermatol, 91:56-61, 1988. With this apparatus skin is fastened between a receptor chamber and a chimney top by a spring clamp. The cells allow a 1.0 cm² portion of epidermis to be exposed to ambient temperature, light, heat and humidity while the dermis is bathed in a 5 ml of receptor solution maintained at 37° C. by water which circulates within a jacket around the lower chamber. The receptor solution was isotonic phosphate buffered saline pH 7.3-7.4 (PBS) with 0.5% Volpo (a nonionic surfactant). Volpo was used to ensure the solubility of the active ingredient in the receptor solution. The receptor solution was continuously stirred by a magnet mounted on a motor. 100 microliter dosing solutions were applied with a calibrated positive-displacement pipettor using disposable pipet tips (Wiretrol, Drummond Scientific Company). The receptor solution was assayed by HPLC at the end of the 24 h study.

Skin surface was always washed with acetone to remove remaining drug 24 h after its application. At the end of 24 hours study the skin was removed from the chamber and placed in 50-ml polypropylene screw-capped centrifuge tubes which had been wrapped with aluminum foil to exclude light. 5 mL of acetonitrile were added, mixed by inversion for 30 minutes and the vials were centrifuged for 5 minutes at 4000 rpm. The organic layer was assayed by HPLC. HPLC was performed on a Hewlett Packard HP 1090 system. A 20 μL aliquot of the supernatant was then injected into a Zorbax SB-C18 column (4.6×75 m, 3.5 μm) equipped with a Zorbax SB-C18 Guard cartridge (4.6×12.5 mm) and operated with aqueous 70% acetonitrile containing 5% glacial acetic acid and 0.02% triethylamine as mobile phase (1 ml/min) and detection at 350 nm. The calibration was linear for 5-1000 ng/ml of sample.

In the final result, when comparing a conventional formula containing retinoic acid dissolved in ethanol and cremophor to the nanoparticulate formula, the amount of retinoic acid in the skin layers is not significantly different between the formulas at a 0.05% and a 0.5% retinoic acid loading level (P=0.05). In addition, efficacy tests using the Rhino Mouse Model (Kligman A M, The effect on rhino mouse skin of agents which influence keratinization and exfoliation. J Invest Dermatol 1979, 73:354-358) have shown that the particulate preparation has similar efficacy to the conventional formula at the 0.05% retinoic acid concentration level. These results are unexpected since tretinoin particulates over one micron in size were previously not believed to cross the stratum corneum (Schaefer H., Penetration and Percutaneous Absorption of Topical Retinoids, in: Retinoids: 10 Years On; Karger Edition, NY, N.Y., p. 17, 1993). However, both skin penetration and efficacy studies indicate that retinoic acid is bioavailable in the tretinoin hydrogel when delivered in this fashion.

When comparing a conventional formula containing retinoic acid dissolved in ethanol and cremophor to a formula using particles, the amount that penetrates the skin as obtained by the Franz cell assay as described earlier is not significantly different at the 0.05% level. Efficacy tests using the Rhino Mouse Model (Kligman A M, The effect on rhino mouse skin of agents which influence keratinization and exfoliation. J Invest Dermatol 1979, 73:354-358) have shown that the particle preparation has similar efficacy to the conventional formula.

In addition, a chitosan hydrogel containing retinol or retinoic acid particles, have been shown to provide a higher degree of fusion with the skin when the particle size of the chitosan matrix is reduced. Such size reduction can be obtained by means of an extrusion using a microfluidizer, or a high pressure homogenizer, as described above.

### EXAMPLE 7

Comparison between Tretinoin Formulas in Terms of Irritation

The vehicle used to carry the active compound has a profound effect on irritation. Ethanol has an LD50 of 3% with irritation generally found at values greater than 5-10% of LD50. Non ionic surfactants have an LD50 less than 1%. Conventional formulas often have concentrations of surfactants exceeding these LD50 values. It is then reasonable to expect conventional tretinoin formulas such as tretinoin emollient creams which contain surfactants and solubilizers to lead to substantial skin irritation. On the other hand, particulates of tretinoin obtained by the methods of the invention are significantly less irritating as shown in the following examples. However, the prior art compositions required these agents for solubilization, and their use could not be avoided.

Preclinical Study involving Gel Formulation Containing Retinoic Acid Particles Obtained Using a Microfluidizer Apparatus to Reduce the Particle Size.

A 3-months preclinical study was undertaken in both mice and rabbits to determine the severity of skin reactions after application of the retinoic acid gel as described above using the Draize test. The animals (40 New Zealand White Rabbits and 140 CD-1 mice) were divided into 5 groups as shown in Table 1, 2 and 3.

The test compound was formulated to include a concentration of 0.05 wt % of retinoic acid in particulate form as illustrated in Example 6 and applied at 100 times and 500 times the human dose (Groups 3 and 4). The vehicle gel and the vehicle gel containing the nanoparticle without retinoic acid (Groups 1 and 2) acted as negative controls whereas a commercial 0.05% cream in a standard emulsion formula at 500 the human dose (Group 5) acted as positive control. As shown in Table 1 in the rabbit study, it was soon apparent that the positive control was too irritating for the animals and three steps were taken to manage the toxicity of the positive control group: (1) the positive control dose was scaled back to 100 times the human dose from 500 the human dose after 10 days of application; (2) a second site of application of the positive control was required while waiting for the first site to heal (about 2 weeks later) and (3) the animals which displayed the greatest discomfort were given an intramuscular injection of buprenorphine (2 out of 8 animals). As shown in Table 3, the nanoparticle delivery system alone did not cause erythema or edema and treatment groups 3 and 4 showed a statistically significant lower irritation and edema level compared to group 5.

TABLE 3
Rabbit study -Average Erythema and Edema
Scores at 10 days post treatment

| | | Average | Average | |
|---|---|---|---|---|
| | | Erythema | Edema | |
| Group Number | | (n = 8) | (n = 8) | Comment |
| 1. | Vehicle | 0.125 | 0 | No erythema/edema |
| 2. | Vehicle + Particles | 0 | 0 | No erythema/edema |
| | (no ATRA) | | | |
| 3. | Vehicle + ATRA | 0.125 | 0 | No erythema/edema |
| | Particles (100× | | | |
| | human dose) | | | |
| 4. | Vehicle + ATRA | 0.875 | 0.25 | Very Slight erythema; |
| | Particles (500× | | | no edema |
| | human dose) | | | |
| 5. | Conventional ATRA | 2.125 | 1.875 | Well defined erythema; |
| | cream (500× human | | | slight edema |
| | dose) | | | |

After treating the second application site with the cream at the lower dose (100 times the human dose) for an additional 14 days the level of erythema and edema significantly exceeded the test compound at 100 and the 500 times the human dose (Table 4).

TABLE 4
Rabbit study -average erythema and edema
scores at 24 days post-treatment

| | | Average | Average | |
|---|---|---|---|---|
| | | Erythema | Edema | |
| Group Number | | (n = 8) | (n = 8) | Comment |
| 1. | Vehicle | 0.125 | 0 | No erythema/ |
| | | | | edema |
| 2. | Vehicle + Particles | 0 | 0 | No erythema/ |
| | (no ATRA) | | | edema |
| 3. | Vehicle + ATRA | 0 | 0 | No erythema/ |
| | Particles (100 times | | | edema |
| | the human dose) | | | |
| 4. | Vehicle + ATRA | 1.75 | 0.75 | Slight erythema; |
| | Particles (500 times | | | very slight |
| | the human dose) | | | edema |
| 5. | Conventional Cream | 2.125 | 1.5 | Well defined |
| | (100 times the human | | | erythema; |
| | dose)* | | | slight edema |

*Applied on second site starting at day 10

In the mice study, at 10 days post treatment the positive control group (Group 5—-Conventional Cream) receiving 100 times the human dose had significantly more erythema than Group 4 treated with 500 times the human dose of test compound (Table 5):

TABLE 5
Mice Study -Average Erythema and Edema
Scores at 10 days post-treatment

| | | Average | Average | |
|---|---|---|---|---|
| | | Erythema | Edema | |
| Group Number | | (n = 28) | (n = 28) | Comment |
| 1. | Vehicle | 0 | 0 | No erythema/ |
| | | | | edema |
| 2. | Vehicle + Particles | 0.07 | 0 | No erythema/ |
| | (no ATRA) | | | edema |
| 3. | Vehicle + ATRA | 0.57 | 0.25 | Very slight |
| | Particles (100 times | | | erythema; |
| | the human dose) | | | no edema |
| 4. | Vehicle + ATRA | 1.0 | 0.46 | Very slight |
| | Particles (500 times | | | erythema; |
| | the human dose) | | | no edema |
| 5. | Conventional Cream | 1.67 | 1.21 | Well defined |
| | (100 times the human | | | erythema; |
| | dose)* | | | slight edema |

The preclinical results indicate that the test compound is significantly less irritating than a conventional retinoic acid cream formula in both rabbit and mice studies. These results strongly suggest the potential for increasing patient compliance in patients undergoing retinoic acid therapy for skin diseases such as acne, photodamage and prevention of melanoma.

### EXAMPLE 8

Long-Term Stability of Particles

The stability of tretinoin in the nanoparticle gel containing 0.05% tretinoin prepared in Example 6 was measured over 203 days. After preparation, the gel was stored under ambient conditions at room temperature (25° C.).

Following preparation of the gel and at periodic intervals afterwards, aliquots of the gel were analyzed by HPLC to determine the concentration of tretinoin remaining. The results were as follows:

| Time (days) | Tretinoin (mg/mL) |
|---|---|
| 0 | 0.51 |
| 96 | 0.50 |
| 203 | 0.50 |

These data demonstrate that there was no appreciable loss of tretinoin in over 200 days. Typically, a significant fraction of the tretinoin would have been lost to oxidation over this period. Thus, the nanoparticles of tretinoin stabilize the tretinoin.

### EXAMPLE 9

Skin Permeability Studies

Skin permeability studies were performed using skin explants with formulations containing trans-retinoic acid at 0.1% concentration in gels containing either the free retinoic acid or the chitosan-entrapped retinoic acid. The apparatus consisted of 6 Franz diffusion cells (PermeGear Inc.) operating in parallel and maintained at a constant temperature of 37° C. Approximately 200 mg/cm² of each formulation containing 0.04 μCi of ³H-ATRA was applied to the epidermal side of the skin sample (1 cm²). Each formulation was tested in triplicate. The dermal surface of the skin was perfused with receptor solution consisting of buffered saline containing 0.05% Volpo (Croda, Inc.). At daily intervals, 500 μL of the receptor solution was sampled to obtain kinetic data. At the end of a 200 hour run a surface wash consisting of 2×500 μL of a 1% acetic acid solution in absolute ethanol was applied to the skin surface. The skin sample was digested overnight in 4 mL of Solvable (Packard Instruments). The entire contents of the receptor volume (5 mL), the surface wash, and digested skin layer were then mixed with Ultima Gold scintillation fluid (Packard Instruments) for ³H counting.

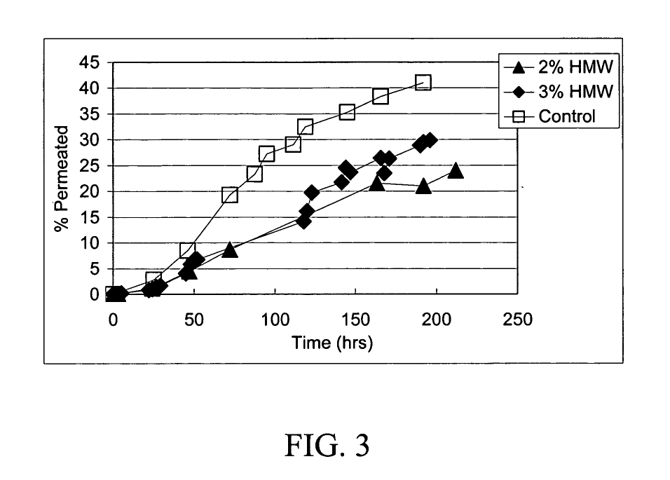

The formulations containing the free retinoic acid delivered a large amount of drug through the skin in the first 50 to 120 hours, while the chitosan-entrapped formulation delivered the retinoic acid at a much slower and constant rate (after an initial lag). The effect of polymer concentration on percutaneous transport was investigated and was found to level off above a biopolymer concentration of over 2% (FIG. 3).

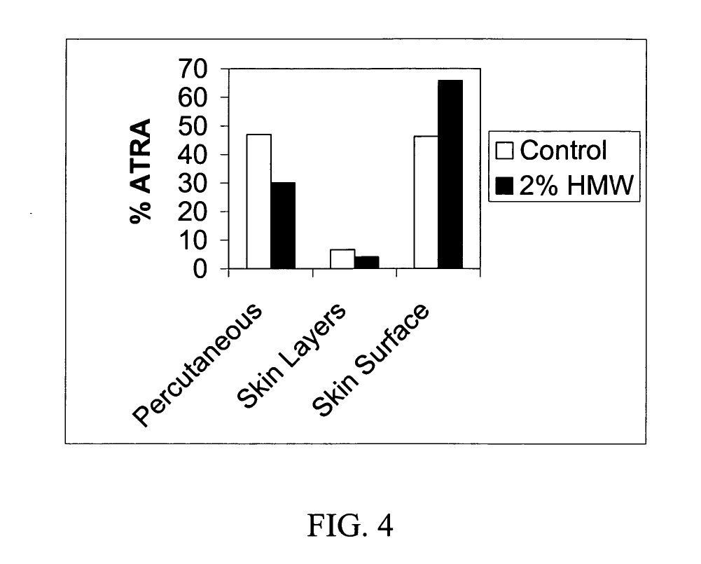

At the end of the skin permeability study, when the distribution of the active in the different skin compartments was evaluated, it was seen that the amount of drug, which penetrated percutaneously from the biopolymer matrix formulation, was 40% lower compared than that obtained using the free drug formula. When the skin layers were evaluated for drug content, however, there was not significant difference (FIG. 4).

These results indicate that the chitosan based delivery system was able to reduce systemic absorption by 40%, but it did not interfere with the amount of retinoic acid taken up by the skin, the site of therapeutic action. Based on these results the chitosan-entrapped retinoic acid formulation should exhibit reduced irritancy in comparison with free retinoic acid.

### EXAMPLE 10

Vitamin E Moisturizing Gel

| | Ingredient | Wt. % |
|---|---|---|
| A | Deionized water | 86.8 |
| | Disodium EDTA | 0.1 |
| | Sodium Alginate (Protanal LF10/60; FMC Biopolymer) | 1.0 |
| | Aloe Barbadensis (Activera ® 100-200C; Active Organics) | 0.5 |
| | Xanthan Gum (Keltrol ® T; CP Kelco) | 0.5 |
| B | Triethanolamine | 0.1 |
| C | Vitamin E Chitosphere ™ | 10.0 |
| D | Preservatives | 1.0 |
| | Total | 100.0 |

The Part A ingredients are weighed into a suitable vessel equipped with a mixer. The mixture is mixed at room temperature until uniform. Part B is added to adjust the pH to 7.0. The Part C ingredient is added separately under vigorous stirring conditions until a homogenous mixture is formed. Part D is added to form the final preparation.

### EXAMPLE 11

Preparation of Tretinoin Hydrogel

Water-insoluble all-trans retinoic acid (ATRA) in the form of solid particles (2 wt %; size less than 100 microns) dispersed in soybean oil (17%) was incorporated into high viscosity chitosan solutions [3 wt % solutions of Protasan UP B 80/500 (FMC Biopolymers Inc.; 755 cps apparent viscosity) in 2.1 wt % glycolic acid and 0.03 wt % sodium hydroxide] by vigorous mixing to form a matrix. The viscosity of the matrix was initially 215,000 cps as measured on a Brookfield LVT viscometer at 25° C. with appropriate spindle at 1.5 rpm. The emulsion was then mixed with a poly(acrylic acid) solution (0.5 wt %) at pH between 5 and 6 and mixed to make a gel.

### EXAMPLE 12

In Vivo Irritation Caused by Retinoid Formulations

The skin irritation caused by various retinoid formulations was assayed. The assay specifically compared two formulations comprising high viscosity chitosan and all-trans-retinoic acid (ATRA), which were prepared by a process similar to that described in Example 11, one with and one without surfactant, and RETIN-A MICRO®, which is a commercially available formulation of ATRA. The website for RETIN-A MICRO® acknowledges that this product may cause “[d]ryness, redness or peeling.”

The assay was conducted based upon the International Organization of Standardization Biological Evaluation of Medical Devices—Part 10: Test for Irritation and Delayed-Type Hypersensitivity, ISO 10993-10, 2002 and General Requirements for the Competence of Testing and Calibration Laboratories, ISO 17025, 1999.

The skin of 24 albino rabbits was prepared for testing. The three test materials (formulations) described above were evaluated using 6 animals per material. Application sites for both test material and control were prepared by clipping the skin of the trunk free of hair within 24 hours before application of the test material. The sites of application were not abraded deliberately or accidentally during preparation. Areas of untreated skin served as the control sites. Animals were weighed prior to the initial application of test material and at the end of the study.

The animals were treated by introducing four different concentrations of the test materials at four separate sites. The animals were exposed to the test materials for 24±2 hours daily for 10±2 days. Test and control sites were shaved closely to the skin prior to the first application and then as often as judged necessary thereafter. The dosing sites were wiped clean with moistened water-for-injection (WFI) gauze sponges and dried before dosing.

The test material formulations for all groups were dosed at 1 g/kg on Day 0, then at 0.5 g/kg for the remainder of the study. The formulations were spread with an applicator to cover the treatment area (dorsum of rabbit), approximately 8-10 cm², avoiding contamination of hair where possible. An Elizabethan collar was placed on each animal. The applicator was wiped clean with a slightly moistened WFI gauze sponge between each animal. Six hours (±15 minutes) after treatment, the Elizabethan collars were removed. The time of collar removal was based upon the group start of treatment.

At the end of each contact period, the application sites were marked. Residual test material was removed by washing with WFI. The application sites were scored 1 hour after washing, prior to the next application. After the last exposure, animals were observed for signs of erythema and edema at 1, 24, 48 and 72 hours. Observations were scored according to the “Classification System for Scoring Skin Reactions,” as shown below:

| | Value |
|---|---|
| Erythema and Eschar Formation | |
| No erythema | 0 |
| Very slight erythema (barely perceptible) | 1 |
| Well defined erythema | 2 |
| Moderate erythema | 3 |
| Severe erythema (beet redness) to slight eschar | 4 |
| formation (injuries in depth) | |
| Total possible erythema score | 4 |
| Edema Formation | |
| No edema | 0 |
| Very slight edema (barely perceptible) | 1 |
| Slight erythema (edges are well defined by | 2 |
| definite raising) | |
| Moderate edema (raised approximately 1 mm) | 3 |
| Severe edema (raised more than 1 mm and | 4 |
| extending beyond area of exposure) | |
| Total possible erythema score | 4 |

Observations were extended when necessary to evaluate reversibility, not to exceed 14 days. For each animal, irritation scores for both erythema and edema at each time were added together. This total was divided by the total number of observations to obtain the irritation score per animal. The irritation scores of each animal were added together and divided by the total number of animals. This value is the Cumulative Irritation Index. The Cumulative Irritation Index was compared to the categories of Cumulative Irritation Index below and the appropriate category was recorded. Scores obtained during the exposures and from the 1, 24, 48 and 72 hour observations after the last exposure were used for calculations. Observations made after 72 hours (to monitor recovery) were not used in the determination.

The test materials were classified based upon their Cumulative Irritation Indices. A test material with a Cumulative Irritation Index of 0 to 0.4 is considered a negligible irritant. A test material with an index of 0.5 to 1.9 is considered a slight irritant. A test material with an index greater than 2.0 and less than 5.0 is considered a moderate irritant. A test material with an index greater than 5.0 is considered a severe irritant. A test material that destroys the structure of intact skin or changes it irreversibly is considered corrosive.

All formulations showed signs of irritation, except the group dosed with the vehicle used for dilution of the formulations. The degree of irritation generally correlated with the potency of the formulations, except in the RETIN-A MICRO® group, where little difference was seen between the sites dosed with the 0.04% and 0.1% formulations. By the fourth day of dosing, all of the animals to which the more potent formulations of RETIN-A MICRO® were administered exhibited moderate irritation (scores of 3 for erythema and 1-2 for edema) with dry, flaky skin, while only half of the animals to which the surfactant-free chitosan formulation exhibited such irritation. All of the animals to which the surfactant-containing chitosan formulation was administered exhibited moderate to severe irritation (scores of 3-4 for erythema and 2 for edema) with dry, flaky skin and, at some sites, fissures with slight bleeding. Notably, three of the animals to which the surfactant-free was administered exhibited only mild irritation at this time. Also, the extent of dry, flaky skin was less frequent and less severe in this group of animals as whole.

No additional test material was administered to the animals that exhibited moderate or severe irritation (i.e., all except 3) on Days 5 and 6; dosing continued in the remaining animals. After the final dose occurring at about Day 10, sign of erythema or edema were noted, but reduced over the 24, 48 and 72 hour observation points. All scores were reversed or were reduced to slight in all animals by the 72 hour observation point. None of the untreated control sites of any animal at any of the observation points showed signs of erythema or edema.

The Cumulative Irritation Index and the appropriate response category for each formulation are listed below:

| Formulation | Strength | Irritation Index |
|---|---|---|
| Vehicle | N/A | 0.2 (negligible) |
| RETIN-A MICRO ® | 0.04% | 3.4 (moderate) |
| | 0.1% | 3.3 (moderate) |
| Surfactant-Containing Chitosan | 0.001% | 2.2 (moderate) |
| | 0.01% | 3.3 (moderate) |
| | 0.05% | 3.8 (moderate) |
| | 0.1% | 4.9 (moderate) |
| Surfactant-Free Chitosan | 0.001% | 0.7 (slight) |
| | 0.01% | 1.0 (slight) |
| | 0.05% | 1.8 (slight) |
| | 0.1% | 2.7 (moderate) |

Based on the Cumulative Irritation Indices and the skin site observations, the surfactant-free formulation was the least irritating of the ATRA formulations. Moreover, this formulation was also well tolerated by the test animals. In contrast, both the surfactant-containing formulation and the RETIN-A MICRO® formulation were moderately irritating at all potencies tested. Thus, the surfactant-free ATRA formulation significantly reduces the irritation and other adverse side effects of ATRA, as compared to a conventional ATRA formulation.

While the invention has been described in detail with reference to certain preferred embodiments thereof, it will be understood that modifications and variations are within the spirit and scope of that which is described and claimed.

What is claimed is:

1. A composition for administration of a water insoluble or slightly water soluble active agent, comprising particles of active agent having a mean diameter of 100 microns or less, wherein said particles are enclosed within droplets of a dispersing agent, wherein said droplets are embedded in a matrix comprising cationic and anionic polymers.

2. A composition for administration of an irritating active agent, comprising particles of active agent having a mean diameter of 100 microns or less, wherein said particles are enclosed within droplets of a dispersing agent, wherein said droplets are embedded in a matrix comprising cationic and anionic polymers, wherein said composition is less irritating than the active agent alone.

3. The composition of claim 1 or 2, wherein the active agent has a solubility of less than 10 mg/mL in the dispersant at 25° C.

4. The composition of claim 1 or 2, wherein the particles are equal to or less than 10 microns in mean diameter.

5. The composition of claim 4, wherein the particles are 1 micron to 10 microns in mean diameter.

6. The composition of claim 1 or 2, wherein the cationic polymer is chitosan.

7. The composition of claim 6, wherein the chitosan is high viscosity chitosan.

8. The composition of claim 6, wherein the composition is obtained under vigorous stirring conditions by (a) forming an emulsion comprising aqueous solutions of a high viscosity chitosan polymer with the active agent particles, dispersed in a suitable dispersing agent, and (b) precipitating the emulsion by complexing with an anionic polymer to form a coacervate complex at the interface between the two polymers.

9. The composition of claim 1 or 2, wherein the pharmaceutical active agent is a retinoid.

10. The composition of claim 9, wherein the retinoid is retinoic acid.

11. The composition of claim 1 or 2, wherein the composition is substantially free of surfactants.

12. The composition of claim 2, wherein the composition has at least 90 percent cell viability in an MTT assay.

13. A method of treating a skin disease or condition, comprising administering the composition of claim 1 to a subject suffering from the skin disease or condition.

14. A method of preparing a hydrogel composition comprising water soluble or slightly water soluble active agent particles, wherein said particles have a mean diameter of less than 100 microns, wherein said particles are dispersed in droplets of a suitable dispersing agent and wherein said droplets are entrapped within a polymer matrix comprising a cationic and an anionic polymer, comprising:
forming an emulsion comprising an aqueous solution of a cationic polymer with the active agent, dispersed in the dispersing agent,
precipitating the emulsion by complexing with an anionic polymer and optionally raising the pH to between 5 and 6, to form a hydrogel.

15. A method of preparing a hydrogel composition comprising water soluble or slightly water soluble active agent particles, wherein said particles have a mean diameter of less than 100 microns, wherein said particles are dispersed in droplets of a suitable dispersing agent and wherein said droplets are entrapped within a polymer matrix comprising a cationic and an anionic polymer, comprising:
creating an emulsion of the active agent suspended in a mixture of the dispersing agent in a aqueous solution of a cationic polymer with a high pressure homogenizer,
adding an anionic polymer to the emulsion to form a hydrogel.
